(19) United States
(12) Patent Application Publication
(10) Pub. No.: US 2008/0125750 A1
(43) Pub. Date: May 29, 2008

(54) Medical catheter removal

(76) Inventor: Henning Arthur Gaissert, Dover, MA (US)

Correspondence Address: FISH & RICHARDSON PC, P.O. BOX 1022, MINNEAPOLIS, MN, 55440-1022, US

(21) Appl. No.: 11/527,865

(22) Filed: Sep. 27, 2006

(51) Int. Cl. A61M 25/00

(52) U.S. Cl. 604/523

(57) ABSTRACT

An apparatus for removing an indwelling catheter from a patient, including a tubular sheath having a first end and a second end, the first end having an aperture for receiving an exterior portion of an indwelling catheter partially inserted into a patient. The sheath has a contracted state and an extended state, the sheath in the extended state being configured to form a sleeve around the catheter. The apparatus includes a coupler having an aperture for receiving the catheter, the coupler being attached to the second end of the sheath, and a one-way valve attached to the coupler, the one-way valve having an aperture dimensioned to accommodate the catheter.

### BACKGROUND OF THE INVENTION

The description relates to removal of medical catheters.

A chest catheter can be used after chest trauma, open heart surgery, operations on the lungs, esophagus, or other organs in the chest, or to treat pneumothorax. The chest catheter can drain air and fluid from the space surrounding the lung to prevent lung collapse or to re-expand a collapsed lung. Negative pressure in the pleural space can help maintain the lung in an expanded state. The negative pressure can be applied to the chest catheter to restore or maintain the lung in the expanded state. When the lung is restored to the expanded state, the chest catheter can be removed.

Removing a chest catheter has risks for the patient and the caregiver. For example, lung collapse (pneumothorax) may occur during removal of the chest catheter. The lung collapse may be caused by air entering the pleural space through the catheter exit site. Once a pneumothorax is found, additional examination may be required, and the patient may be required to remain hospitalized for a longer period of time. To reduce this risk, the caregiver may quickly pull out the chest catheter causing pain to the patient. The caregiver may be sprayed or splashed with blood or infectious body fluids. The patient or caregiver clothing or the hospital bed may be soiled or contaminated by blood or body fluids, requiring a change of clothes or bed sheets.

### SUMMARY

In general, in one aspect, an apparatus includes a tubular sheath having a first end and a second end, the first end having an aperture for receiving an exterior portion of an indwelling catheter partially inserted into a patient, the sheath having a contracted state and an extended state, the sheath in the extended state being configured to form a sleeve around the catheter. The apparatus includes a coupler having an aperture for receiving the catheter, the coupler being attached to the second end of the sheath, and a one-way valve attached to the coupler, the one-way valve having an aperture dimensioned to accommodate the catheter.

Implementations of the apparatus may include one or more of the following features. The sheath includes a plastic film. The one-way valve is at least partially positioned within the sheath. The one-way valve is configured to provide unidirectional fluid and air communication from a space inside the patient to a space surrounded by the sheath. The apparatus includes an adhesive attached to the first end of the folded film, the adhesive being covered by a liner. The second end of the film has a breakpoint to allow detachment from the coupler. The coupler includes an O-ring. The sheath is configured to transition from its contracted state to an extended state by extending bellows. The one-way valve includes a flutter valve. The one-way valve includes a Heimlich valve. The apparatus includes the catheter.

In general, in another aspect, a method of removing an indwelling catheter from a patient body. The method includes pulling the catheter from the patient, and while pulling the catheter, passing the catheter through a one-way valve, and extending a sheath to enclose a portion of the catheter previously in the patient, the one-way valve allowing unidirectional flow of air or fluid from inside the patient body to an exterior environment.

Implementations of the apparatus may include one or more of the following features. The method includes attaching a first end of the sheath to an exterior portion of the catheter to form a seal between the sheath and an exterior circumference of the catheter. The method includes, while pulling the catheter, passing the catheter through a coupler having a first side attached to the patient and a second side attached to the one-way valve. The method includes, while pulling the catheter, passing the catheter through a coupler having a first side attached to the patient and a second side attached to the sheath. The method includes, when the catheter is completely withdrawn from the patient, detaching a first end of the sheath from the coupler, and closing the first end. The method includes maintaining one end the catheter exterior to the patient body connected to a drainage reservoir while pulling the catheter. The one-way valve includes a flutter valve.

In general, in another aspect, a method of reducing the likelihood of lung collapse when removing a chest catheter from a patient. The method includes passing an exterior portion of the chest catheter through a one-way valve, attaching the one-way valve to the skin of the patient, pulling the chest catheter from the patient, while pulling the chest catheter, passing the chest catheter through the one-way valve, and completely pulling the chest catheter out of the patient while keeping the one-way valve attached to the patient skin, the one-way valve allowing fluid and air to egress from the body of the patient and preventing fluid or air to enter the body.

Implementations of the apparatus may include one or more of the following features. The method includes extending a sheath to enclose a portion of the catheter previously in the patient. Attaching the one-way valve to the skin of the patient includes attaching a coupler to the skin, the one-way valve being attached to the coupler.

### DESCRIPTION OF DRAWINGS

FIG. 1 is a diagram of a chest catheter removal device.

FIGS. 2-5 are diagrams illustrating use of the chest catheter removal device in removing a chest catheter.

FIG. 6 is a diagram of a chest catheter removal device.

### DESCRIPTION

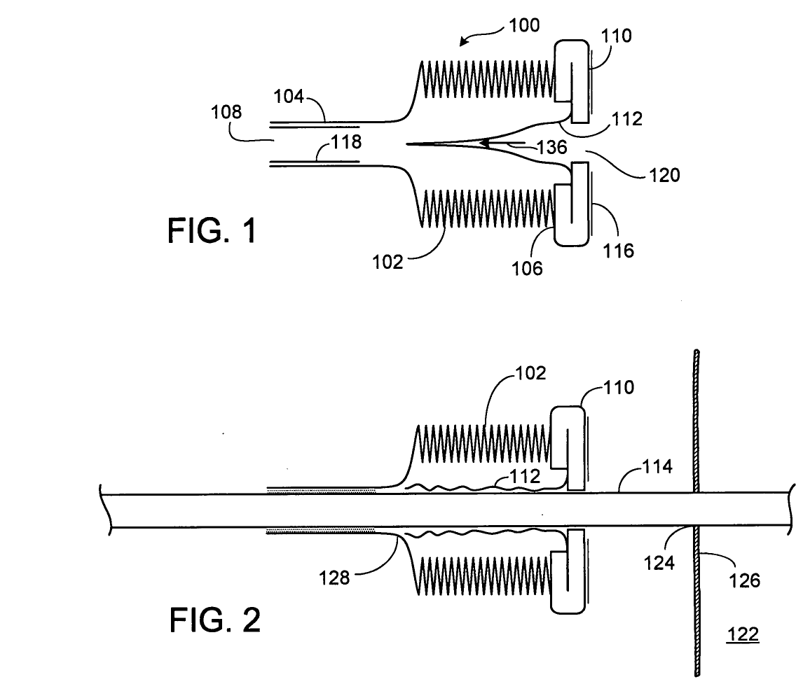

Referring to FIGS. 1 and 2, a chest-catheter removal device 100 includes a tubular sheath 102 having a first end 104 and a second end 106. The first end 104 has an aperture 108 for receiving an exterior portion of a chest catheter 114 (or chest tube) that is partially inserted in a chest 122 of a patient. FIG. 1 shows the sheath 102 in its contracted state. The sheath 102 can be extended to form a tubular sleeve around the chest catheter 114 as the chest catheter 114 is pulled out of the patient. The removal device 100 includes a coupler 110 that is attached to the patient before removing the tube. The coupler 110 is made of flexible material so that it can conform to the contour of the patient body, and includes an aperture that is slightly larger than the diameter of the catheter 114. An example of the coupler 110 is an O-ring.

A flutter valve 112 is attached to the coupler 110 and positioned within the sheath 102. The flutter valve 112 extends from the coupler 110 into the sheath 102. The flutter valve 112 is a one-way valve that allows fluids or air to flow in one direction 136. An example of the flutter valve 112 is a Heimlich valve.

The sheath 102 is a tubular, pliable, foldable, plastic (e.g., PVC, polyethylene, or other Latex-free) film that, in its extended state, is longer than the portion of the chest catheter 114 inserted in the patient and of slightly larger diameter than the chest catheter 114. In some examples, the tubular sheath 102 folds into an accordion-like shape.

An adhesive 118 is provided on the inside surface at the first end 104 of the tubular sheath 102. The adhesive 118 is protected by a liner, which is peeled off just prior to use.

The second end 106 of the tubular sheath 102 is attached to the coupler 110 in a way that allows easy detachment of the tubular sheath 102 from the coupler 110. For example, there may be a break point, or perforation, at the second end 106 of the tubular sheath 102.

The coupler 110 has an adhesive 116 that is covered by a protective liner. The adhesive 116 is strong enough to allow the coupler 110 to form an air-tight seal at the patient skin, but also weak enough to allow the coupler 110 to be detached from the patient without damaging the skin.

FIGS. 2-5 show how the chest catheter removal device 100 is used.

Referring to FIG. 2, the chest catheter 114is inserted into the patient chest 122 at an insertion site 124. While in its folded state, the tubular sheath 102 is slipped over the chest catheter 114 before the chest catheter 114 is connected to a fluid collection reservoir (e.g., Pleurovac). The liner of the adhesive at the first end 104 of the tubular sheath 102 is removed, and the first end 104 is secured to the chest catheter 114, forming a seal 128 to prevent fluid or air from passing through the first end 104. The first end 104 remains attached to the chest catheter 114 when the catheter 114 is in the patient body and throughout the process of removing the chest catheter 114.

While the chest catheter 114 is inserted in the patient's chest, the device 100 surrounds the chest catheter 114 and the tubular sheath 102 is maintained in its folded configuration. When the chest catheter 114 needs to be removed, the coupler 110 is moved towards the patient. Doing so extends the tubular sheath 102 and unfolds it until the coupler 110 reaches the skin 126 around the insertion site 124 of the chest catheter 114. The protective liner is removed from the adhesive film 116 of the coupler 110, and the coupler 110 adheres to the skin 126.

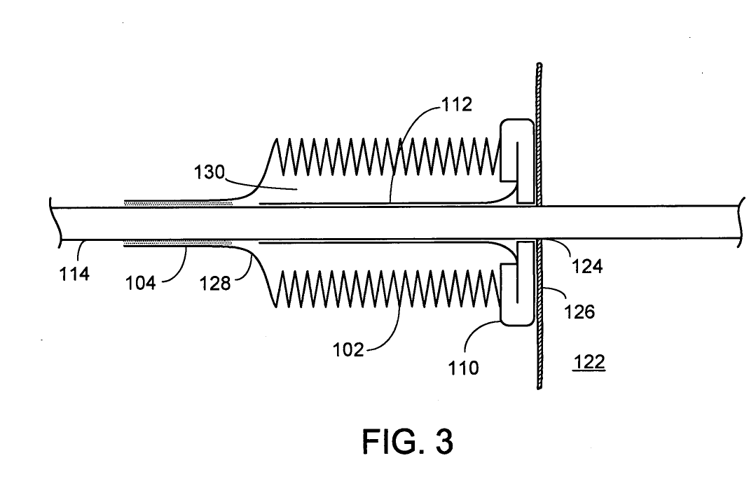

Referring to FIG. 3, when the coupler 110 is attached to the skin 126, the portion of the chest catheter 114 extending from the insertion site 124 to the first end 104 is entirely contained in the tubular sheath 102. The removal device 100 can have a small chamber 130 that is formed between the chest catheter 114 and the inner side of the tubular sheath 102. The small chamber 130 forms an extension of the pleural space of the patient and can collect body fluids that drip from the catheter 114 as the catheter 114 is being pulled out. Although the small chamber 130 contains a small amount of air, the amount is not likely to result in a significant pneumothorax if suctioned into the chest.

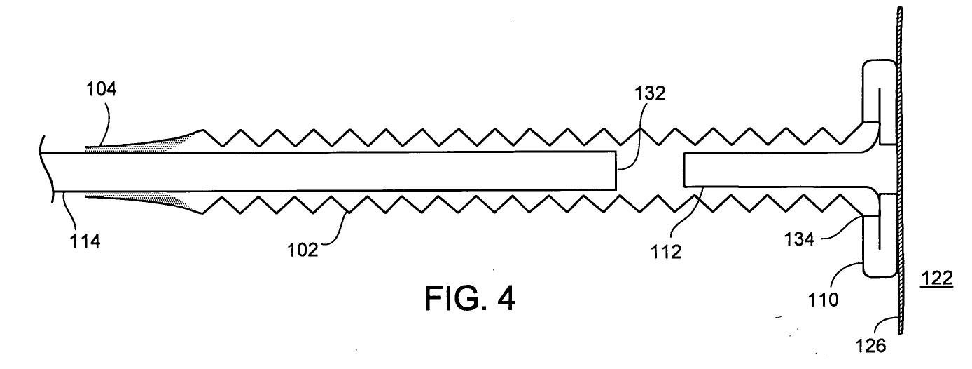

Referring to FIG. 4, the chest catheter 114 is slowly removed from the pleural space of the patient until the tip 132 of the catheter 114 has passed several inches beyond the end of the flutter valve 112. The tubular sheath 102 is then separated from the coupler 110 at a pre-fabricated breakpoint 134.

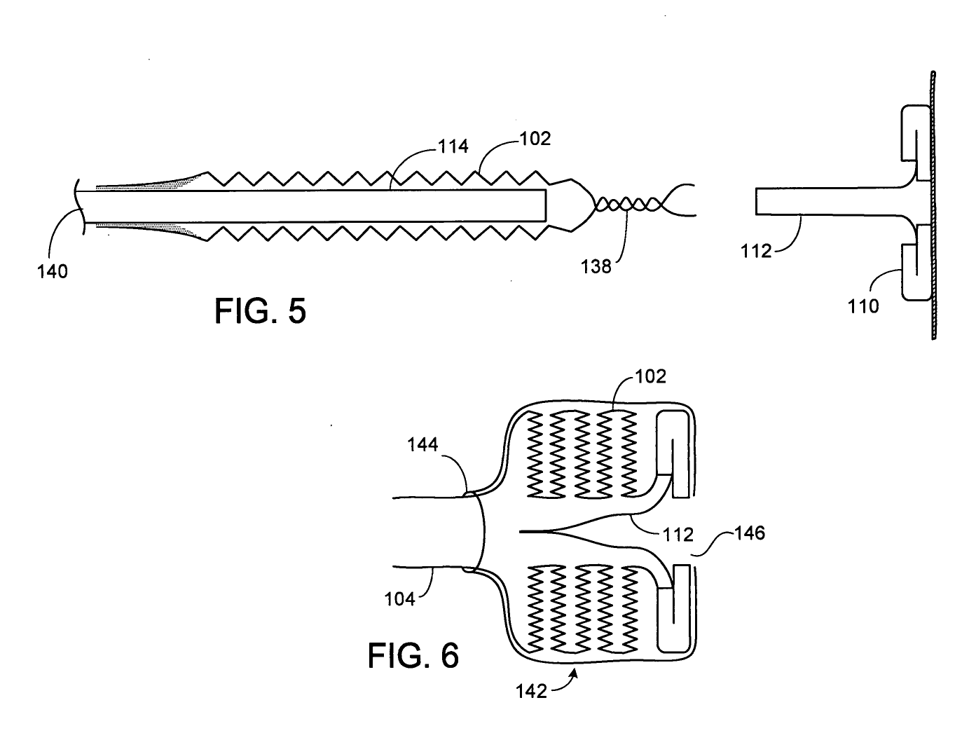

Referring to FIG. 5, when the tubular sheath 102 is separated from the coupler 110, the flutter valve 112 prevents air from entering the chest, but allows air or fluid to escape from the chest. The tubular sheath 102 containing the chest catheter 114 can be closed by a plastic clamp, a rubber ring, or an adhesive flap to form a sealed end 138 to prevent leakage of any residual body liquid or contaminated air borne particles. The other tip 140 of the catheter 114 remains connected to the chest drainage reservoir (for example, Pleurovac) to prevent leakage of fluids or contaminated air borne particles. The chest catheter 114 along with the device 100 is then disposed of. The flutter valve 112, which is still attached to the patient's skin 126, is covered with gauze.

The device 100 can reduce the likelihood of lung collapse (pneumothorax). Lung collapse may be caused by air entering the pleural space through the insertion site during or after removal of the chest tube. Once a pneumothorax is found on a radiograph after removal of the chest tube, additional radiographs may be ordered, or the discharge of the patient may be delayed. Side effects caused by removal of chest catheters may require patients to remain hospitalized after surgery on their chests. Complications that may arise in connection with removal of chest tubes are described in “Are chest radiographs routinely necessary following thoracostomy tube removal?” by Pacharn et al., Pediatric Radiology, vol. 32, number 2, February 2002, and “What keeps postpulmonary resection patients in hospital?” by Bardell T. et al., Canadian respiratory journal, 2003 March; 10(2):86-9. Using the device 100 can also reduce the likelihood of requiring additional radiographs, delayed discharge from the-hospital, and their associated costs.

Using the chest tube removal device 100 can reduce the likelihood of spray or splash contamination by infectious body fluids when the chest catheter 114 is being withdrawn from the patient. The device 100 can reduce the likelihood of soiling of any nearby clothing or bed sheets.

The device 100 is small and unobtrusive until needed for removal of the chest catheter 114. The tubular sheath 102 can then be unfolded and extended at the time of chest catheter removal. Because the sheath 102 does not need to be sturdy in its extended state, lightweight materials that are impervious to liquid can be used for the tubular sheath 102.

Because there is reduced likelihood of lung collapse and contamination by the body fluid, the chest catheter 114 can be removed slowly and deliberately, without haste, potentially decreasing patient discomfort and complication rate.

The chest catheter removal device 100 can be used in different kinds of medical procedures that require the use of chest catheter, for example, coronary artery bypass grafting, lung lobectomy and other lung resections, video-assisted thoracic surgery, esophageal resection, spontaneous lung collapse, and chest trauma. The device 100 can be used with other types of catheters or tubes, such as endotracheal tubes, nasogastric tubes, and catheters inserted into other locations of the body.

The device 100 can be designed to have different sizes in order to accommodate catheters or tubes of different diameters. The chest catheter 114 can be, for example, sizes 12 to 24 French for pediatric use and 24 to 36 French for adult use. The catheter 114 and the device 100 may be packaged together and provided as a medical kit. Alternatively, the device 100 can be provided separately from the chest catheter 114, so that the device 100 is slipped on to the chest catheter 114 after the catheter is inserted into the patient body.

Although some examples have been discussed above, other implementations and applications are also within the scope of the following claims. For example, the tubular sheath 102 can be made of a different material, and can be folded in ways that differ from those described above. The sheath 102 can be configured like a bellows whose length can be adjusted. The sheath 102 can have various thicknesses. Various types of flutter valves can be used. The coupler 110 can have a shape different from what is described above. The sheath 102 can have different colors. Referring to FIG. 6, the sheath 102 can be folded irregularly and occupy a small space in its contracted state. A film 142 may form a tubular enclosure that encloses the sheath 102 in its contracted state. The tubular enclosure has an opening 144 to accommodate the first end 104 of the sheath 102. The tubular enclosure has another opening 146 to accommodate the catheter 114. The film 142 remains intact prior to removal of the catheter 114 to maintain the sheath 102 in its contracted state. The film 142 can have perforations so that it can easily be torn open to allow the sheath 102 to be extended when the catheter 114 is to be removed.

What is claimed is:

1. An apparatus comprising:
a tubular sheath having a first end and a second end, the first end having an aperture for receiving an exterior portion of an indwelling catheter partially inserted into a patient, the sheath having a contracted state and an extended state, the sheath in the extended state being configured to form a sleeve around the catheter;
a coupler having an aperture for receiving the catheter, the coupler being attached to the second end of the sheath; and
a one-way valve attached to the coupler, the one-way valve having an aperture dimensioned to accommodate the catheter.

2. The apparatus of claim 1 wherein the sheath comprises a plastic film.

3. The apparatus of claim 1 wherein the one-way valve is at least partially positioned within the sheath.

4. The apparatus of claim 1 wherein the one-way valve is configured to provide unidirectional fluid communication from a space inside the patient to a space surrounded by the sheath.

5. The apparatus of claim 1, further comprising an adhesive attached to the first end of the folded film, the adhesive being covered by a liner.

6. The apparatus of claim 1 wherein the second end of the film has a breakpoint to allow detachment from the coupler.

7. The apparatus of claim 1 wherein the coupler comprises an O-ring.

8. The apparatus of claim 1 wherein the sheath is configured to transition from its contracted state to an extended state by extending bellows.

9. The apparatus of claim 1 wherein the one-way valve comprises a flutter valve.

10. The apparatus of claim 1 wherein the one-way valve comprises a Heimlich valve.

11. The apparatus of claim 1, further comprising the catheter.

12. A method of removing an indwelling catheter from a patient body, comprising:
pulling the catheter from the patient; and
while pulling the catheter,
passing the catheter through a one-way valve, and
extending a sheath to enclose a portion of the catheter previously in the patient, the one-way valve allowing unidirectional fluid flow from inside the patient body to an exterior environment.

13. The method of claim 12, further comprising attaching a first end of the sheath to an exterior portion of the catheter to form a seal between the sheath and an exterior circumference of the catheter.

14. The method of claim 12, further comprising, while pulling the catheter, passing the catheter through a coupler having a first side attached to the patient and a second side attached to the one-way valve.

15. The method of claim 12, further comprising, while pulling the catheter, passing the catheter through a coupler having a first side attached to the patient and a second side attached to the sheath.

16. The method of claim 15, further comprising, when the catheter is completely withdrawn from the patient, detaching a first end of the sheath from the coupler, and closing the first end.

17. The method of claim 12, further comprising maintaining one end the catheter exterior to the patient body connected to a drainage reservoir while pulling the catheter.

18. The method of claim 12 wherein the one-way valve comprises a flutter valve.

19. A method of reducing likelihood of lung collapse when removing a chest catheter from a patient, the method comprising:
passing an exterior portion of the chest catheter through a one-way valve;
attaching the one-way valve to the skin of the patient;
pulling the chest catheter from the patient;
while pulling the chest catheter, passing the chest catheter through the one-way valve; and
completely pulling the chest catheter out of the patient while keeping the one-way valve attached to the patient skin, the one-way valve allowing fluid and air to egress from the body of the patient and preventing fluid or air to enter the body.

20. The method of claim 19, further comprising extending a sheath to enclose a portion of the catheter previously in the patient.

21. The method of claim 19, wherein attaching the one-way valve to the skin of the patient comprises attaching a coupler to the skin, the one-way valve being attached to the coupler.
